(19) United States
(12) Patent Application Publication
(10) Pub. No.: US 2022/0088421 A1
(43) Pub. Date: Mar. 24, 2022

(54) Air Purification System

(71) Applicant: John H. Moore, Wynnewood, OK (US)

(72) Inventor: John H. Moore, Wynnewood, OK (US)

(21) Appl. No.: 17/147,038

(22) Filed: Jan. 12, 2021

Related U.S. Application Data
(60) Provisional application No. 63/082,099, filed on Sep. 23, 2020.

(51) Int. Cl. A62B 18/04; A61L 9/014; B01D 46/00; B01D 39/20; A62B 7/10; A62B 18/00

(52) U.S. Cl. CPC A62B 18/045; A61L 9/014; B01D 46/0036; B01D 46/0043; B01D 46/0023; B01D 39/2055; B01D 2273/30; A62B 7/10; A62B 18/003; A61L 2209/14; A61L 2209/22; B01D 2279/65; B01D 2267/40; B01D 46/0028

(57) ABSTRACT

An air purifying breathing system for providing filtered air to a user. The air purifying breathing system comprises a headgear that is not self-contained and is open under a face shield to let air flow out of the headgear. A fan component moves ambient air into the system and across a filtering component. The filtering component comprises a first and second filter, each filter using different filter media. After the air is filtered, it exits the filtering component and into the headgear at the top for distribution to the user. The filtered air flows past the user and out the opening under the face shield. The slight positive pressure from the airflow prevents the inflow of unfiltered air into the headgear. The filtering component may be attached to or integrated into the headgear or may be retained by a vest or harness.

### CROSS-REFERENCE TO RELATED APPLICATION

The present application claims priority to, and the benefit of, U.S. Provisional Application No. 63/082,099, which was filed on Sep. 23, 2020 and is incorporated herein by reference in its entirety.

### FIELD OF THE INVENTION

The present invention generally relates to a purification system for breathing air, and more specifically to a wearable portable breathing air filtration system to protect a user from respiratory threats. Accordingly, the present specification makes specific reference thereto. However, it is to be appreciated that aspects of the present invention are also equally amenable to other like applications, devices and methods of manufacture.

### BACKGROUND

Airlines, theme parks, sports venues, cruise ships, restaurants, and other public venues are shutting down and going out of business due to the fear of contracting contagious diseases. Germs, viruses, bacteria, microbes, and other pathogens are tiny living things found throughout the natural world. These organisms are constantly present and persist in the air that we breathe. A pathogen is a micro-organism that has the potential to cause disease. Inhaling these pathogens or introducing them into mucus membranes that exist in the eyes, nose, and mouth can lead to serious illness or even death.

Viruses are by far the most common cause of infectious diseases acquired in indoor environments causing considerable impact on human health. Transmission of respiratory viruses are assumed to occur predominantly directly from person to person. COVID-19 is a virus caused by a coronavirus called SARS-CoV-2. Older adults and people who have severe underlying medical conditions like heart or lung disease or diabetes seem to be at higher risk for developing more serious complications from COVID-19 illness. The disease is thought to spread mainly from person to person, mainly through respiratory droplets produced when an infected person coughs or sneezes. These droplets can land in the mouths or noses of people who are nearby or possibly be inhaled into the lungs. Spread is more likely when people are in close contact with one another and exposed to exhaled air from an infected individual.

One way of combating these inhaled viruses if the use of protective face coverings, such as face masks. There are many types of masks with varying levels of effectiveness. Typically, a large number of people must all wear masks simultaneously and properly to be somewhat effective at reducing disease transmission. Mask wearing compliance unfortunately is a challenge with many people outright refusing to wear any kind of face covering. This leaves individuals desiring protection at risk.

There are higher level protective masks, such as N95 respirators. The Centers for Disease Control and Prevention (CDC) does not recommend that the general public wear N95 respirators to protect themselves from respiratory diseases, including COVID-19. Those respirators are considered critical supplies that must be reserved for health care workers and other medical first responders, as recommended by current CDC guidance. An N95 respirator is a respiratory protective device designed to achieve a very close facial fit and very efficient filtration of airborne particles. Note that the edges of the respirator are designed to form a seal around the nose and mouth. To be effective, individuals need to be fit-tested to ensure that the user is protected. These masks have a thick filter medium and are not very comfortable for long periods of use.

Accordingly, there is a long felt need in the art for an improved air purifying breathing system. Moreover, there is also a long felt need in the art for an air purifying breathing system attached to or integrated into a headgear that is a positive-pressure system, and that prevents the inflow of unfiltered air into the headgear. Finally, there is also a long felt need in the art for an improved air purifying breathing system that is relatively inexpensive to manufacture and both safe and easy to use.

In this manner, the improved air purification system of the present invention accomplishes all of the forgoing objectives, thereby providing an easy solution to keep people safe during a viral outbreak or other health emergency. A primary feature of the present invention is the ability to use positive pressure in conjunction with filtration techniques to keep breathing air safe. Finally, the air purification system allows individuals to be protected against infection without the need to rely on the compliance of others following safety guidelines or laws.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an air purifying breathing system. The air purifying breathing system is a positive-pressure system that is not self-contained. The air purifying breathing system comprises a headgear, a fan component, and a filtering component. The headgear is typically a helmet comprising a face shield that is not sealed at the neck opening allowing for the outflow of air. The fan component is a battery powered motor-driven fan which pulls ambient air in through an intake and forces the air under pressure through the air purifying breathing system. The fan component may be attached to or integrated into an exterior of the headgear.

The filtering component comprises a first filter and a second filter. The first and second filters are in-line with and in communication with the fan component. The fan component generates an airflow that is forced through the first and second filters to purify the air. The first filter is a charcoal filter that may be attached to or integrated into the exterior of the headgear downstream from the fan component. The second filter is a high efficiency particulate filter that may be attached to or integrated into the exterior of the headgear downstream from the first filter.

The air purifying breathing system further comprises an air distributor. The air distributor is positioned within an interior of the headgear downstream of the second filtering component. The air distributor is configured to direct the airflow from a top of the headgear toward the face shield for inhalation by the user. The airflow creates a slight positive pressure within the headgear that exits at the neck opening and prevents unfiltered ambient air from entering the headgear through the neck opening. This is advantageous as the air purifying breathing system eliminates breathing resistance that would be inherent in using a negative-pressure respirator or other filter mask.

In an additional embodiment, an air purifying breathing system comprises a headgear, a fan component, a first filtering component, and a second filtering component. The headgear is typically a helmet comprising a face shield that is not sealed at the neck opening allowing for the outflow of air. The fan component is a battery powered motor-driven fan which pulls ambient air in through an intake and forces the air under pressure through the air purifying breathing system. The fan component is not attached to the headgear.

The first and second filtering components are in-line with and in communication with the fan component. The fan component generates an airflow that is forced through the first and second filtering components to purify the air. The first filtering component is a charcoal filter that is not attached to the headgear. The first filtering component is connected with the fan component via an inflow tubing. The second filtering component is a high efficiency particulate filter that may be attached to or integrated into an exterior of the headgear. An outflow tubing connects the second filtering component to the first filtering component.

The air purifying breathing system further comprises a bodywear component. The bodywear component may be a vest. The bodywear component may comprise a harness and a plurality of pockets. The pockets are configured to retain replacement batteries, replacement filters, the fan component, or the first filtering component. Alternatively, the harness is configured to retain the fan component and the first filtering component.

The air purifying breathing system further comprises an air distributor. The air distributor is positioned within an interior of the headgear downstream of the second filtering component. The air distributor is configured to direct the airflow from a top of the headgear toward the face shield for inhalation by the user. The airflow creates a slight positive pressure within the headgear that exits at the neck opening and prevents unfiltered ambient air from entering the headgear through the neck opening. This is advantageous as the air purifying breathing system eliminates breathing resistance that would be inherent in using a negative-pressure respirator or other filter mask.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side perspective view of one potential embodiment of an air purifying breathing system of the present invention and in accordance with the disclosed architecture;

FIG. 2 illustrates a front perspective view of one potential embodiment of the air purifying breathing system of the present invention and in accordance with the disclosed architecture;

FIG. 3 illustrates a rear perspective view of one potential embodiment of the air purifying breathing system of the present invention and in accordance with the disclosed architecture;

FIG. 4 illustrates a perspective view of one potential embodiment of an air purifying breathing system of the present invention in accordance with the disclosed architecture;

FIG. 5 illustrates a side perspective view of an airflow generated through one potential embodiment of a headgear of the air purifying breathing system of the present invention in accordance with the disclosed architecture;

FIG. 6 illustrates a rear perspective view of one potential embodiment of the air purifying breathing system of the present invention and in accordance with the disclosed architecture; and

FIG. 7 illustrates a front perspective view of one potential embodiment of the air purifying breathing system of the present invention and in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is an air purification system designed to provide a clean, safe, filtered air supply for the general public. A portable breathing system comprises a vest, a helmet, and a face shield that a user can wear in public spaces. A battery-operated air blower intakes ambient air and forces it through a hose across a replaceable activated charcoal filter. The vest is configured to hold component parts including spare batteries. The portable breathing system further comprises a replaceable high efficiency particulate air (HEPA) filter. The helmet is pushed out and away from the face and cut off at the bottom near the chin to accommodate talking and eating.

The air hose connects the blower to the activated charcoal filter, while a second air hose connects the activated charcoal filter to the HEPA filter housed in the helmet. The HEPA filter cartridges can be interchanged and replaced as needed. The air hose is also secured to the vest of via a hook and loop flap covering. It can also hold fully charged spare blower batteries which can be replaced without having to remove the vest. The batteries can maintain air delivery for eight hours or more. The blower can intake air and deliver clean, filtered air to the wearer while he/she is in a public space. The air purification system is particularly useful for large attended events where mask wearing compliance is potentially an issue.

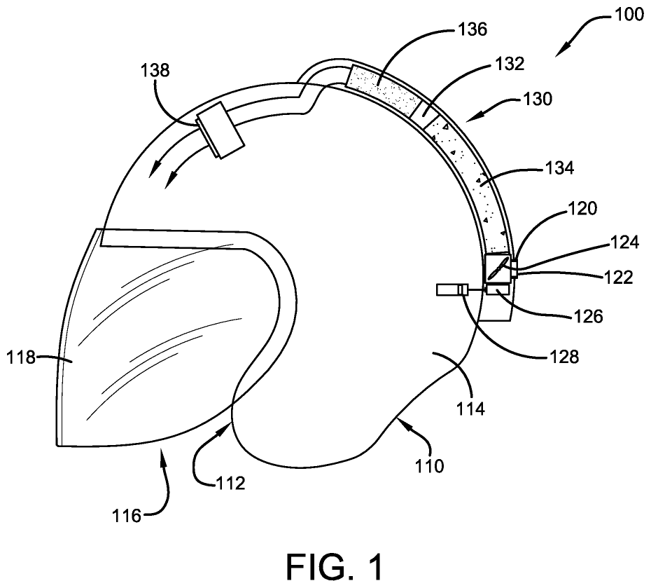

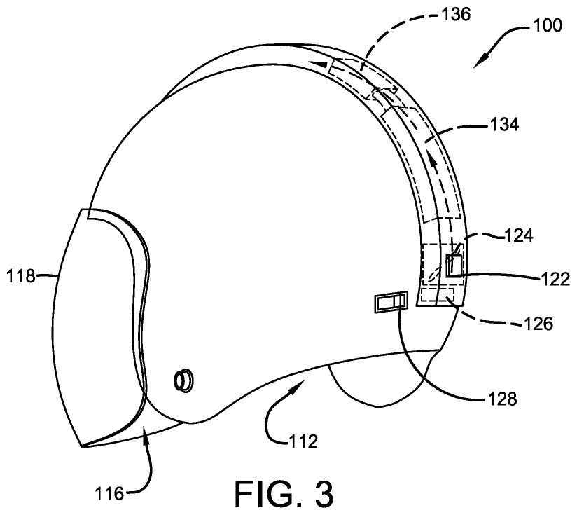

Referring initially to the drawings, FIGS. 1-3 illustrate an air purifying breathing system 100. The air purifying breathing system 100 is a positive-pressure system that is not self-contained. The air purifying breathing system 100 comprises a headgear 110, a fan component 120, and a filtering component 130. The headgear 110 is typically a helmet. The headgear 110 comprises an interior 112, an outside 114, and a face shield 118. The headgear 110 is not sealed at a neck opening 116 allowing for the outflow of air from the interior 112 through the neck opening 116. The face shield 118 can be pivotally attached to the headgear 118 so that it can flip up for eating, or the face shield 118 may be fixed in place.

The fan component 120 comprises a motor-driven fan 124 and an air intake 122. A rechargeable battery 126 powers the motor-driven fan 124 which is controlled by a power switch 128. The motor-driven fan 124 pulls ambient air in through the air intake 122 and forces the air under pressure through the air purifying breathing system 100. The motor-driven fan 124 may be a blower to achieve the proper pressure requirements for the system 100. The motor-driven fan 124 may be a variable speed fan for adjusting airflow. The fan component 120 may be attached to or integrated into an outside 114 of the headgear 110.

The filtering component 130 comprises a first filter 134 and a second filter 136. The first and second filters 134 and 136 are in-line with and in communication with the fan component 120. The fan component 120 generates an airflow that is forced through the first and second filters 134 and 136 along an airflow path 132 to purify the air. The purified air then proceeds into the interior 112 of the headgear 110. The first filter 134 is an activated charcoal filter that may be attached to or integrated into the outside 114 of the headgear 110 downstream from the fan component 120. The second filter 136 is a high efficiency particulate filter that may be attached to or integrated into the outside 114 of the headgear 110 downstream from the first filter 134. Both the first and second filters 134 and 136 are replaceable.

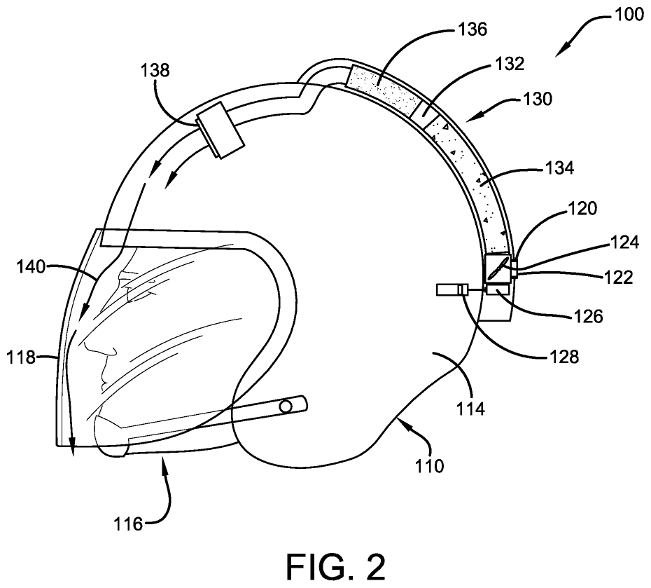

The air purifying breathing system 100 further comprises an air distributor 138. The air distributor 138 is positioned within the interior 112 of the headgear 110 downstream of the second filtering component 136. The air distributor 138 is configured to direct an airflow 140 from a top of the headgear 110 toward the face shield 118 for inhalation by the user as illustrated in FIG. 2. The airflow 140 creates a slight positive pressure within the headgear 110 that exits at the neck opening 116 and prevents unfiltered ambient air from entering the headgear 110 through the neck opening 116 during use. This is advantageous as the air purifying breathing system 100 eliminates breathing resistance that would be inherent in using a negative-pressure respirator or other filter mask.

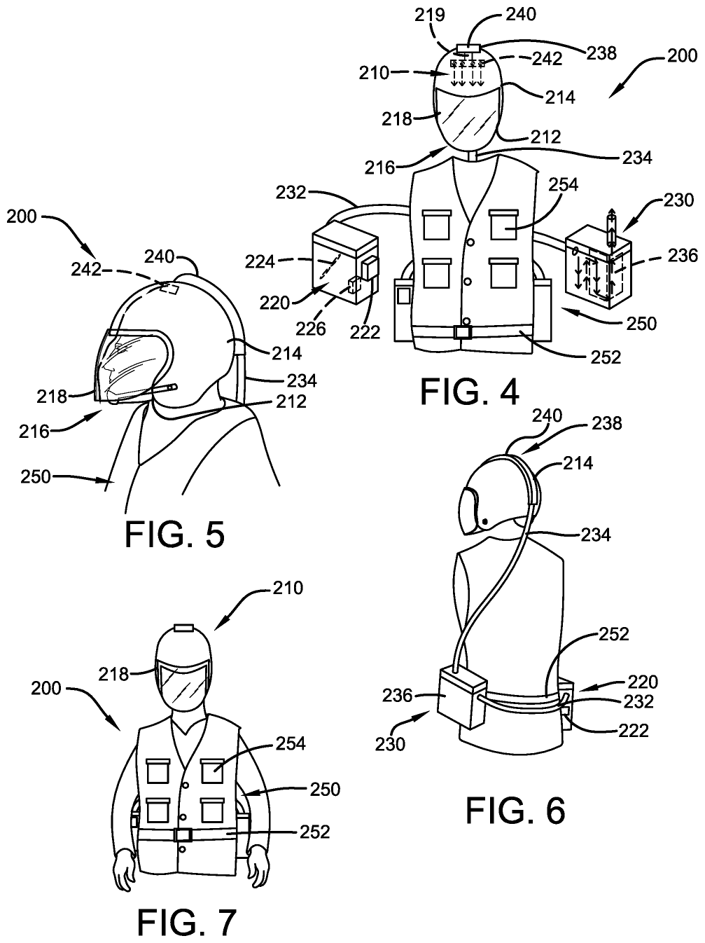

In an additional embodiment as illustrated in FIGS. 4-7, an air purifying breathing system 200 comprises a headgear 210, a fan component 220 a first filtering component 230, and a second filtering component 238. The headgear 210 is typically a helmet. The headgear 210 comprises an interior 212, an outside 214, and a face shield 218. The headgear 210 further comprises an air inlet 219 for treated air to enter the interior 212 of the head gear 210. The headgear 210 is not sealed at a neck opening 216 allowing for the outflow of air from the interior 212 through the neck opening 216 allowing for the outflow of air.

The fan component 220 comprises a motor-driven fan 224 and an air intake 222. A rechargeable battery 226 powers the motor-driven fan 224 which is controlled by a power switch (similar to 128). The motor-driven fan 224 pulls ambient air in through the air intake 222 and forces the air under pressure through the air purifying breathing system 200. The motor-driven fan 224 may be a variable speed fan for adjusting airflow. The fan component 220 is not attached to the headgear 210.

The first and second filtering components 230 and 238 are in-line with and in communication with the fan component 220. The first and second filtering components 230 and 238 are both replaceable filters. The fan component 220 generates an airflow that is forced through the first and second filtering components 230 and 238 to purify the air. The purified air then proceeds into the interior 212 of the headgear 210. The first filtering component 230 is located external to the headgear 210 and comprises a first filter media 236. The first filter media 236 is typically an activated charcoal filter that is replaceable. Alternatively, the first filter media 236 may be removed if not needed, with the second filtering component 238 still in place to provide filtering. The first filtering component 230 is connected with the fan component 220 via an inflow tubing 232. The second filtering component 238 is integrated into or attached to the headgear 210. The second filtering component 238 comprises a second filter media 240. The second filter media 240 is typically a high efficiency particulate filter. The second filtering component 238 is connected to the first filtering component 230 via an outflow tubing 234. The connection may be made at a base of the back of the headgear 210, or anywhere along the headgear 210 as desired.

The air purifying breathing system 200 further comprises a bodywear component 250. The bodywear component 250 may be a vest. The bodywear component 250 may comprise a harness 252 and a plurality of pockets 254. The harness 252 may be a belt, holster, hook and loop flap system, or the like and may also retain the inflow tubing 232. The pockets 254 are configured to retain replacement batteries, replacement filters, the fan component 220, or the first filtering component 230. Alternatively, the harness 252 is configured to retain the fan component 220 and the first filtering component 230.

The air purifying breathing system 200 further comprises an air distributor 242. The air distributor 242 is positioned within the interior 212 of the headgear 210 downstream of the second filtering component 238. The air distributor 242 is configured to direct the airflow into the headgear 210 from a top of the headgear 210 toward the face shield 218 for inhalation by the user. The airflow creates a slight positive pressure within the headgear 210 that exits at the neck opening 216 and prevents unfiltered ambient air from entering the headgear 210 through the neck opening 216.

Notwithstanding the forgoing, the improved air purifying breathing system 100 and 200 can by any suitable size, shape, and configuration as is known in the art without affecting the overall concept of the invention, provided that it accomplishes the above stated objectives. One of ordinary skill in the art will appreciate that the shape and size of the air purifying breathing system 100 and 200 and its various components, as show in the FIGS. are for illustrative purposes only, and that many other shapes and sizes of the air purifying breathing system 100 and 200 are well within the scope of the present disclosure. Although dimensions of the air purifying breathing system 100 and 200 and its components (i.e., length, width, and height) are important design parameters for good performance, the air purifying breathing system 100 and 200 and its various components may be any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An air purifying breathing system comprising:
a headgear;
a fan component;
a filtering component comprising a first filter and a second filter in communication with the fan component; and
wherein the fan component is configured to generate an airflow through the first and second filters and into an interior of the headgear.

2. The air purifying breathing system of claim 1, wherein the fan is attached to an outside of the headgear.

3. The air purifying breathing system of claim 1, wherein the headgear is a helmet that is not self-contained.

4. The air purifying breathing system of claim 1, wherein the airflow creates a positive-pressure within the interior of the headgear.

5. The air purifying breathing system of claim 1, wherein the airflow is adjustable.

6. The air purifying breathing system of claim 1, wherein the first filter is a charcoal filter.

7. The air purifying breathing system of claim 1, wherein the second filter is a high efficiency particulate air filter.

8. The air purifying breathing system of claim 1, wherein the first and second filters are replaceable.

9. An air purifying breathing system comprising:
a headgear;
a fan component;
a first filtering component external to the headgear in communication with the fan component;
a second filtering component in communication with the first filtering component; and
wherein the fan component is configured to generate an airflow through the first and second filtering components and into an interior of the headgear.

10. The air purifying breathing system of claim 9, wherein the second filtering component is integrated into the headgear.

11. The air purifying breathing system of claim 9, wherein the first filtering component comprises a first filter media and the second filtering component comprises a second filter media.

12. The air purifying breathing system of claim 11, wherein the first filter media is a replaceable charcoal filter.

13. The air purifying breathing system of claim 11, wherein the second filter media is a replaceable high efficiency particulate air filter.

14. The air purifying breathing system of claim 9, wherein the airflow moves into the helmet via an air distributer positioned within the headgear downstream of the second filtering component.

15. The air purifying breathing system of claim 9, wherein the air distributer is configured to direct the airflow toward a face shield of the headgear.

16. The air purifying breathing system of claim 9 further comprising a bodywear component for retaining the fan component and the first filtering component.

17. The air purifying breathing system of claim 16, wherein the bodywear component comprises a harness and a plurality of pockets.

18. An air purifying breathing system comprising:
a helmet;
a fan component;
a first filtering component comprising a charcoal filter in communication with the fan component via an inflow tubing;
a second filtering component comprising a high efficiency particulate air filter in communication with the first filtering component via an outflow tubing;
a bodywear component for retaining the fan component and the first filtering component; and
wherein the fan component is configured to generate an airflow through the first and second filtering components and into an interior of the headgear.

19. The air purifying breathing system of claim 18, wherein the airflow moves into the helmet via an air distributer positioned within the headgear downstream of the second filtering component and is directed toward a face shield of the helmet.

20. The air purifying breathing system of claim 18, wherein the bodywear component is a vest comprising a plurality of pockets.
